(19) United States
(12) Patent Application Publication
(10) Pub. No.: US 2002/0051801 A1
(43) Pub. Date: May 2, 2002

(54) Compositions for preparing water-in-oil microemulsions

(76) Inventors: Pauline Abu-Jawdeh, London (GB); David Martin, Liverpool (GB)

Correspondence Address: FAY, SHARPE, FAGAN, MINNICHI & McKEE, LLP, Seventh Floor, 1100 Superior Avenue, Cleveland, OH, 44114-2518, US

(21) Appl. No.: 09/947,021

(22) Filed: Sep. 5, 2001

Related U.S. Application Data
(63) Continuation of application No. PCT/GB00/00800, filed on Mar. 6, 2000.

(30) Foreign Application Priority Data
Mar. 6, 1999 (GB) 9905099.9
Jul. 17, 1999 (GB) 9916754.6

(51) Int. Cl. C10L 1/32; A61K 7/00

(52) U.S. Cl. 424/401; 44/301

(57) ABSTRACT

There is provided a composition for preparing a water-in-oil emulsion, wherein the average droplet size of the water phase of the water-in-oil emulsion is no greater than 01 μm. In a preferred embodiment of the invention, the oil is a fuel.

The present invention relates to a composition. In a particular, the present invention relates to a composition for preparing a water-in-oil emulsion, wherein the average droplet size of the water phase is no greater than 0.1 μm. More particularly, the invention relates to water-in-fuel emulsions.

Lubricants used in the production of metals etc. are known to be flowable systems which can be assigned to one of the following three categories.

Neat oils: are as the name suggests base oil that incorporates specialist additives to overcome wear, foaming etc. These lubricants are the most lubricious giving good surface finishes to produced pieces. However, they are also the poorest at cooling the tool/metal interface. This means that the machining rate is much reduced in comparison to soluble oils. Overall neat oils have little in the way of disadvantages other than a lack of coolancy, as they are easy to handle, have a long sump life, can be reconditioned, and are easy to filter and dispose of as and when necessary.

Soluble oils: consist of a concentrate, which is diluted with water by up to 50 times. As they are predominantly water they have a very high degree of coolancy. However, this is at the expense of lubricity and though the metal can be worked very quickly the surface finish is generally poor. Due to their high water content the possibility of both corrosion and bacterial growth arises requiring additional additives such as corrosion inhibitors and biocides. The latter being liable to cause dermatitis thus making handling more of a problem. Disposal of these oils is also very difficult since they cannot be easily incinerated. They are generally demulsified, which is a very expensive procedure.

Water-in-oil emulsions: have been used to give a lubricant that has properties somewhere between the neat and soluble oils. However, these oils are usually unstable and have many of the same problems as the soluble oils. Attempts have been made previously to formulate stable invert emulsions e.g. WO 92/07053, however, these emulsions although having a much reduced droplet size are inherently unstable. This leads to separation problems and also both corrosion and bacterial growth.

Fuels: are defined as hydrocarbon feedstock for powering an engine or other such machinery. The invention uses all fuel types i.e. diesel, kerosene, petrol (leaded or unleaded) and mixtures thereof.

The use of fuel emulsions has been undertaken previously by many applicants. However, the emulsions formed are of a large water droplet size giving a milky appearance. These emulsions require a number of secondary additives such as corrosion inhibitors and bactericides to overcome addition of the water phase. These emulsions due to their large water droplet size also exhibit instability that leads to fuel/water separation. Naturally, this is unwelcome as it may lead to problems with not only machine failure but also problems with loss of production in say a diesel-powered generator.

The present invention describes a composition that allows that formation of translucent microemulsions having a water droplet size of less than 0.1 μm and preferably less than 0.01 μm. This small droplet size not only gives an appearance with is more aesthetically pleasing to the user but offers several major advantages over the present systems. The translucency imparted due to the small droplet size negates the need for both corrosion inhibitors and bactericides. An improvement in the lubricity of the emulsions is also gained probably due to the surfactants, which help to stabilise the emulsion. These systems are also thermodynamically stable by nature and will therefore not have the inherent instability of typical systems.

The advantages seen in these systems will not deter from the advantages of using water-in-fuel emulsions i.e. cleaner emissions, reduced particulate matter and improved combustion rates (leading to better fuel consumption). WO 98/50139 describes the use of microemulsions in several industrial lubricant applications including cutting oils, hydraulic fluids, gear oils and grinding fluids. The composition for preparing the microemulsion comprises i) a fatty acid amine ethoxylate ii) C₆-C₁₅ alcohol ethoxylate; and optionally iii) tall oil fatty acid amine.

The present invention addresses problems of the prior art.

Aspects of the present invention are defined in the appended claims.

The present invention provides new lubricants based on wafer-in-oil emulsions and compositions for preparing the same wherein the oil may be an oil or fuel as defined below. The droplets of the water phase of the emulsion have an average droplet size of no greater than 0.1 μm. These emulsions are clear translucent emulsions. Thus in a further aspect the present invention provides a composition for preparing a water-in-oil emulsion, wherein the emulsion is a clear translucent emulsion. Any reference in the present specification to “a water-in-oil emulsion, wherein the average droplet size of the water phase of the water-in-oil emulsion is no greater than 0. 1 μm”, is analogous to the term “a water-in-oil emulsion wherein the emulsion is a clear translucent emulsion”.

The present emulsions have high lubricity and improved cooling properties without the problems of corrosion or bacterial growth.

An important area of use for the new water-in-oil lubricants is in the rolling and drawing processes of metal production, although the present invention is not limited to this application area.

In one aspect the present invention provides a composition for preparing an emulsion combining the cooling properties of the added water with the lubricity of the oil continuous phase in such a manner that a clear translucent fluid is obtained that has the properties of a neat oil with additional coolancy. Whilst giving these benefits the emulsions of this invention exhibit none of the disadvantages associated with conventional soluble or invert fluids i.e. bacterial growth, corrosion, reduced sump life

The present invention provides a composition for preparing a stable emulsion. The emulsion is of a continuous oil or fuel phase in which water droplets, having an average droplet size of no greater than<0.1 μm are dispersed. The resultant clear translucent emulsion is thermodynamically stable and offers both high lubricity and cooling properties.

Thus, a first aspect of the invention relates to a composition for preparing a water-in-oil emulsion, wherein the average droplet size of the water phase of the water-in-oil emulsion is no greater than 0.1 μm.

The second aspect of the invention relates to a composition as defined in the first aspect wherein the oil is a fuel.

As discussed above, lubricant emulsions need to be present whilst machining metal in order to produce products that are as high a quality as possible in as short a space of time as possible. This requires that the lubricant must possess a high degree of lubricity to impart good surface finish to the product. The cooling action of the lubricant dictates how quickly the metal can be worked. As discussed earlier neat oils fail to produce material quickly due to their inherent lack of coolancy. Whilst the soluble and invert emulsions overcome this lack of coolancy they have inherent problems of their own; these being bacterial growth in the fluid and potential problems with corrosion.

The present invention provides a sufficiently high water content fluid that, due to the extremely small droplet size, cannot support microbial growth.

Other than in the operating examples, or where otherwise indicated, all numbers expressing quantities of ingredients used herein are to be understood as modified in all instances by the term “about”.

The emulsion of the present invention may be prepared from base oils disclosed in WO 98/50139. In this aspect a lubricant of high ecological acceptability can readily be achieved offering alternate methods of disposal to the industry.

Preferably, the oil is selected from an ester type oil, a mineral oil, a synthetic type oil, and mixtures thereof, and particularly a fuel as described in the second aspect of the invention.

In accordance with the first aspect of the invention, the mixture ratios of the oil and water phases of the emulsion can be varied depending on the application of the emulsion. Generally speaking, the oil phase comprises at least about 50% by weight, based on the total of the fluid phases, oil and water. Preferably the oil phase comprises between about 50 and 60% by weight. Generally speaking, the oil phase comprises no greater than about 90% by weight or about 95% by weight. (Each percentage by weight is based on the total of the fluid phases oil and water).

In accordance with the second aspect of the present invention, the mixture ratios of the fuel and water phases of the emulsion can be varied depending on the application of the emulsion. Generally speaking, the fuel comprises at least about 80% by weight, based on the total of the fluid phases, fuel and water. Generally speaking, the fuel phase comprises no greater than about 90% by weight or about 95% by weight. (Each percentage by weight is based on the total of the fluid phases fuel and water).

The emulsifying agent components may be chosen from a wide range of emulsifying agents known in the art to be useful in the formation of emulsion lubricant compositions. Examples of such emulsifying agents include alcohol ethoxylates, phenol alkoxylates, poly(oxyalkylene) glycols, poly(oxyalkylene) fatty acid esters, amnine alkoxylates, poly(alkyl) succinimides, poly(alkenyl) succinimides, fatty acid esters of sorbitol and glycerol, fatty acid salts, sorbitan esters, poly(oxyalkylene) sorbitan esters, fatty amine alkoxylates, poly(oxyalkylene) glycol esters, fatty acid amides, fatty acid amide alkoxylates, fatty amines, quaternary amines, alkyloxazolines, alkenyloxazolines, imidazolines, alkyl-sulphonates, alkylarylsulphonates, alkylsulfosuccinates, alkylphosphates, alkenylphosphates, phosphate esters, derivatives and mixtures thereof.

More preferable emulsifying agents include alcohol ethoxylates, poly(oxyalkylene) glycols, amine alkoxylates, poly(alkyl) succinimides, sorbitan esters and fatty amine alkoxylates and mixtures thereof.

The emulsifier composition is chosen to minimise the amount of emulsifier to form a microemulsion for a given base fluid. Typically, the composition comprises between 5 and 30% by weight of emulsifier. Preferably, between 5 and 20% and more preferably between 5 and 10%.

Among the preferred emulsifying agents are the alcohol ethoxylates, fatty acid amines, fatty acid amides, ethoxylated fatty acid amines, ethoxylated fatty acid amides and fatty acid esters.

Preferred Embodiments of the First (Water-in-Oil) Aspect

In general, the compositions for preparing water-in-oil or water-in-fuel emulsions comprise a C₆-C₁₅ alcohol ethoxylate, an amine ethoxylate and a polyisobutylsuccinimide or sorbitan ester.

In a preferred embodiment of the first aspect the present invention may provide a composition which comprises the following: (i) 4 parts C₆ - C₁₅ alcohol ethoxylate; (ii) 1 part amine ethoxylate; and (iii) 1 part polyisobutylsuccinimide.

In a further preferred aspect the present invention may provide a composition which comprises the following: (i) 3 parts amine ethoxylate; (ii) 1 part fatty acid amine; and (iii) 1 part polyisobutylsuccinimide.

In an alternative embodiment of the first aspect, the present invention may provide a composition which comprises the following: (i) 2 parts C₆ -C₁₅ alcohol ethoxylate; (ii) 2 part fatty acid amine ethoxylate; and (iii) 1 part sorbitan ester.

In a further preferred aspect the present invention may provide a water-in-oil emulsion comprising (i) 20 parts water; (ii) 80 parts an ester type oil; and (iii) a composition as defined herein, in amount of 17 parts by volume relative to the total oil and water.

In a further preferred aspect the present invention may provide a water-in-oil emulsion comprising (i) 30 parts water; (ii) 70 parts a mineral oil; and (iii) a composition as defined herein, in amount of 23 parts by volume relative to the total oil and water.

In a further preferred aspect the present invention may provide a water-in-oil emulsion comprising (i) 20 parts water; (ii) 80 parts a synthetic type oil; and (iii) a composition as defined herein, in amount of 16 parts by volume relative to the total oil and water.

The present invention may be utilised in, among others, the industrial lubricants applications and is suited to all uses within that application area.

Preferred Embodiments of the Second (Water-in-Fuel) Aspect

In a further embodiment of the invention, the oil is a fuel.

Preferably, the fuel in the present invention is selected from diesel, kerosine, petrol (leaded or unleaded) and mixtures thereof.

Preferably, the composition for preparing a water-in-fuel emulsion comprises:

(a) a C₆-C₁₅ alcohol ethoxylate, polyisobutylsuccinimide and a sorbiton ester or ethylene glycol; or

(b) a C₆-C₁₅ alcohol ethoxylate, sorbiton ester and butoxy ethanol.

In a further preferred aspect the present invention may provide a composition for preparing a water-in-fuel emulsion which comprises the following: (i) 240 parts C₆ -C₁₅ alcohol ethoxylate; (ii) 20 parts sorbitan ester; and (iii) 1 part polyisobutylsuccinimide.

In a further preferred aspect the present invention may provide a composition for preparing a water-in-fuel emulsion which comprises the following: (i) 200 parts C₆ -C₁₅ alcohol ethoxylate; (ii) 50 parts ethylene glycol; and (iii) 1 part polyisobutylsuccinimide.

In a further preferred aspect the present invention may provide a composition for preparing a water-in-fuel emulsion which comprises the following: (i) 2 parts C₆ -C₁₅ alcohol ethoxylate; (ii) 1 part butoxyethanol; and (iii) 1 part sorbitan ester.

In a further preferred aspect the present invention may provide an emulsion comprising (i) 10 parts water; (ii) 90 parts diesel fuel; and (iii) a composition as defined herein, in amount of 14 parts by volume relative to the total fuel and water.

In a further preferred aspect the present invention may provide an emulsion comprising (i) 10 parts water; (ii) 90 parts unleaded petrol; and (iii) a composition as defined herein, in amount of 10 parts by volume relative to the total fuel and water.

In a further preferred aspect the present invention may provide an emulsion comprising (i) 10 parts water; (ii) 90 parts diesel fuel; and (iii) a composition as defined herein, in amount of 12 parts by volume relative to the total fuel and water.

An important area of use for the new water-in-fuel emulsions of the present invention is in the heavy duty diesel engine market, particularly trucks, buses and other heavy duty transport vehicles, although the present invention is not limited to this application area.

The water phase used can be taken directly from the local water supply.

The composition may comprise additional components. These additional components may be incorporated to improve anti-wear Or extreme pressure properties or act as an antifreeze, for example, ethylene glycol. The requirement to add additional components may be dictated by the application area in which the invention is used. Suitable additional components, and the requirement therefor depending on application area, will be apparent to those skilled in the art.

The present invention will now be described only by way of example.

### EXAMPLES

As described above, reference to “a water-in-oil emulsion wherein the emulsion is a clear translucent emulsion” is analogous to the term “a water-in-oil emulsion, wherein the average droplet size of the water phase of the water-in-oil emulsion is no greater than 0.1 μm”. In the present examples emulsions were visually inspected.. Those which were clear and translucent were considered to have an average droplet size of the water phase of the water-in-oil emulsion of no greater than 0.1 μm.

### Example 1

A composition suitable for combining oil with water was prepared by adding the following components in the quantities stated:

4 parts C₆ -C₁₅ alcohol ethoxylate

1 part amine ethoxylate

1 part polyisobutylsuccinimide

The components were gently mixed to form an homogenous solution.

### Example 2

A composition suitable for combining oil with water was prepared by adding the following components in the quantities stated:

3 parts amine ethoxylate

1 part fatty acid amine

1 part polyisobutylsuccinimide

The components were gently mixed to form an homogenous solution.

### Example 3

A composition suitable for combining oil with water was prepared by adding the following components in the quantities stated:

2 parts C₆ -C₁₅ alcohol ethoxylate

2 part fatty acid amine ethoxylate

1 part sorbitan ester

The components were gently mixed to form an homogenous solution.

### Example 4

The composition from Example 1 was used to combine 80 parts of a ester base oil with 20 parts water. The composition was introduced to the oil and water from a burette. The resulting fluid was gently mixed until a clear translucent fluid was observed. The resulting fluid remains stable after more than one year.

### Example 5

The composition from Example 2 was used to combine 70 parts of a mineral base oil with 30 parts water. The composition was introduced to the oil and water from a burette. The resulting fluid was gently mixed until a clear translucent fluid was observed. The resulting fluid remains stable after more than one year.

### Example 6

The composition from Example 3 was used to combine 80 parts of a synthetic base oil with 20 parts water. The composition was introduced to the oil and water from a burette. The resulting fluid was gently mixed until a clear translucent fluid was observed. The resulting fluid remains stable after more than one year.

### Example 7

The fluids from examples 4,5 and 6 have all been subjected to industry standard tests for anti-wear properties, microbial growth, corrosion and anti-foaming properties. All of the fluids demonstrated comparable anti-wear properties to the base fluid from which they were prepared. No microbial growth, corrosion or excessive foaming was observed in any of the fluids.

### Example 8

The fluids from examples 4,5 and 6 were subjected to evaluation of their heat capacity in relation to the base oil from which they were prepared. In all cases the heat capacity was significantly higher in the microemulsions than the straight base fluids. This indicates a higher capacity for the fluid to cool the metal when being worked.

### Example 9

The fluids from examples 4,5 and 6 were subjected to corrosion tests using aluminium test material. This test is particularly relevant for fluids that are intended for use in the rolling oils market. The aluminium was immersed in the fluid and subjected to varying pressures and temperatures (up to 500 psi and 250° C.). In all cases no corrosion was observed on the test materials.

### Example 10

A comparable test to that in example 9 was undertaken using a commercial soluble oil and an invert macroemulsion. In both cases corrosion was observed on the aluminium test pieces.

### Example 11

To demonstrate the ease with which the microemulsion fluids can be disposed a sample of waste material from a machine trial was used as a fuel material in a heating system. The fluid was used with no clean up and found to give no problems to the heating system. Naturally this would not be possible using soluble or water mix fluids due to their high water content.

### Example 12

The microemulsion fluids prepared in the previous examples have been formed using all conventional base fluid types used in the lubricant industry. These being:

Mineral Oils

Naphthenic Oils

Paraffin Oils

Ester Oils

Glycol's

Synthetic Oils

Linear Alpha Hydrocarbons

### Example 13

A composition suitable for combining fuel with water was prepared by adding the following components in the quantities stated:

240 parts C₆ -C₁₅ alcohol ethoxylate

10 parts sorbitan ester

1 part polyisobutylsuccinimide

The components were gently mixed to form a homogenous solution.

### Example 14

A composition suitable for combining fuel with water was prepared by adding the following components in the quantities stated:

200 parts C₆ -C₁₅ alcohol ethoxylate

50 parts ethylene glycol

1 part polyisobutylsuccinimide

The components were gently mixed to form a homogenous solution.

### Example 15

A composition suitable for combining fuel with water was prepared by adding the following components in the quantities stated:

2 parts C₆ -C₁₅ alcohol ethoxylate

1 part butoxyethanol

1 part sorbitan ester

The components were gently mixed to form a homogenous solution.

### Example 16

The composition from Example 13 was used to combine 90 parts of a diesel fuel with 10 parts water. The composition was introduced to the fuel and water from a burette. The resulting fluid was gently mixed until a clear translucent fluid was observed. The resulting fluid remains stable after more than one year.

### Example 17

The composition from Example 14 was used to combine 90 parts of unleaded petrol with 10 parts water. The composition was introduced to the fuel and water from a burette. The resulting fluid was gently mixed until a clear translucent fluid was observed. The resulting fluid remains stable after more than one year.

### Example 18

The composition from Example 15 was used to combine 90 parts of diesel fuel with 10 parts water. The composition was introduced to the fuel and water from a burette. The resulting fluid was gently mixed until a clear translucent fluid was observed. The resulting fluid remains stable after more than one year.

### Example 19

The fluids from Examples 16, 17 and 18 have all been subjected to industry standard tests for anti-wear properties, microbial growth, corrosion and anti-foaming properties. All of the fluids demonstrated comparable anti-wear properties to the base fluid from which they were prepared. No microbial growth, corrosion or excessive foaming was observed in any of the fluids.

### Example 20

The fluids from Examples 16, 17 and 18 were subjected to evaluation of their heat capacity in relation to the base fuel from which they were prepared. In all cases the heat capacity was significantly higher in the microemulsions than the straight fuel.

### Example 21

The fluids from Examples 16, 17 and 18 were subjected to carbon residue tests as outlined in BS EN590. All were within the specifications laid out in the standard document.

### Example 22

The fluids from Examples 16, 17 and 18 were subjected to lubricity evaluation using both the High Frequency Reciprocating Rig (HFRR) AND THE Ball on Cylinder Test (BOCLE). The fluids all demonstrated better lubricity using the BOCGLE test than diesel alone whilst giving wear values<400 μm for the HFRR test (these latter values being well within specification).

### Example 23

The diesel-water emulsion of Example 15 was used to run a diesel engine in a simple test drive. No adverse changes were noted in the performance of the vehicle

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. A composition for preparing a water-in-oil emulsion, wherein the average droplet size of the water phase of the water-in-oil emulsion is no greater than 0.1 μm.

2. A composition according to claim 1 comprising selected from alcohol ethoxylates, phenol alkoxylates, poly(oxyalkylene) glycols, poly(oxyalkylene) fatty acid esters, amine alkoxylates, poly(alkyl) succinimides, poly(alkenyl) succinimides, fatty acid esters of sorbitol and glycerol, fatty acid salts, sorbitan esters, poly(oxyalkylene) sorbitan esters, fatty amine alkoxylates, poly(oxyalkylene) glycol esters, fatty acid amides, fatty acid amide alkoxylates, fatty amines, quaternary amines, alkyloxazolines, alkenyloxazolines, imidazolines, alkylsulphonates, alkylarylsulphonates, alkylsulfosuccinates, alkylphosphates, alkenylphosphates, phosphate esters, derivative and mixtures thereof.

3. A composition according to claim 1 or 2 which comprises the following:
(i) 4 parts C₆-C₁₅ alcohol ethoxylate;
(ii) 1 part amine ethoxylate; and
(iii) 1 part polyisobutylsuccinimide.

4. A composition according to claim 1 or 2 which comprises the following:
(i) 3 parts amine ethoxylate;
(ii) 1 part fatty acid amine; and
(iii) 1 part polyisobutylsuccinimide.

5. A composition according to claim 1 or 2 which comprises the following:
(i) 2 parts C₆-C₁₅ alcohol ethoxylate;
(ii) 2 part fatty acid amine ethoxylate; and
(iii) 2 part sorbitan ester.

6. A composition for preparing a water-in-oil emulsion according to claims 1 or 2, wherein the oil is a fuel.

7. A composition according to claim 6 which comprises the following:
(i) 240 parts C₆-C₁₅ alcohol ethoxylate;
(ii) 20 parts sorbitan ester
(iii) 1 part polyisobutylsuccinimide.

8. A composition according to claim 6 which comprises the following:
(i) 200 parts amine ethoxylate;
(ii) 50 parts ethylene glycol; and
(iii) 1 part polyisobutylsuccinimide.

9. A composition according to claim 6 which comprises the following:
(i) 2 parts C₆-C₁₅ alcohol ethoxylate;
(ii) 1 part butoxyethanol; and
(iii) 1 part sorbitan ester.

10. A water-in-oil emulsion, wherein the average droplet size of the water phase of the water-in-oil emulsion is no greater than 0.1 μm.

11. An emulsion according to claim 10 comprising:
(i) water;
(ii) an oil; and
(ii) a composition as defined in any one of claims 1 to 5.

12. An emulsion according to claim 11 wherein the oil is selected from an ester type oil, a mineral oil, a synthetic type oil, and mixtures thereof.

13. An emulsion according to claim 11 comprising:
(i) 20 parts water;
(ii) 80 parts an ester type oil; and
(iii) a composition as defined in any one of claims 1 to 5, in amount of 17 parts by volume relative to the total oil and water.

14. An emulsion according to claim 11 comprising:
(i) 30 parts water;
(ii) 70 parts a mineral oil; and
(iii) a composition as defined in any one of claims 1 to 5, in amount of 23 parts by volume relative to the total oil and water.

15. An emulsion according to claim 11 comprising:
(i) 20 parts water;
(ii) 80 parts a synthetic type oil; and
(iii) a composition as defined in any one of claims 1 to 5, in amount of 16 parts by volume relative to the total oil and water.

16. An emulsion according to claim 10 comprising:
(i) water;
(ii) a fuel; and
(ii) a composition as defined in any one of claims 6 to 9.

17. An emulsion according to claim 16 wherein the fuel is selected from diesel, unleaded petrol, leaded petrol, kerosene and mixtures thereof.

18. An emulsion according to claim 16 comprising:
(i) 10 parts water;
(ii) 90 parts diesel fuel; and
(iii) a composition as defined in any one of claims 6 to 9, in amount of 14 parts by volume relative to the total fuel and water.

19. An emulsion according to claim 16 comprising:
(i) 10 parts water;
(ii) 90 parts petrol (unleaded); and
(iii) a composition as defined in any one of claims 6 to 9, in amount of 10 parts by volume relative to the total fuel and water.

20. An emulsion according to claim 16 comprising:
(i) 10 parts water;
(ii) 90 parts diesel fuel; and
(iii) a composition as defined in any one of claims 6 to 9, in amount of 12 parts by volume relative to the total fuel and water.

21. A process for the preparation of a water-in-oil emulsion, wherein the average droplet size of the water phase of the water-in-oil emulsion is no greater than 0.1 μm. The process comprising the step of contacting a mixture of oil and water with a composition as defined in any of claims 1 to 9, such that the water-in-oil emulsion is formed and such that the average droplet size of the water phase of the water-in-oil emulsion is no greater than 0.1 μm.

22. A water-in-oil emulsion obtainable by or obtained by a process as defined in claim 21, wherein the average droplet size of the water phase of the water-in-oil emulsion is no greater than 0.1 m.

23. A water-in-oil emulsion according to any one of claims 10 to 15 or 22 for use as an industrial lubricant.

24. A water-in-oil emulsion according to any one of claims 16 to 22 for use as a hydrocarbon feedstock for machinery or engines.

25. Use of a composition as defined in any one of claims 1 to 9 to form a water-in-oil emulsion, wherein the average droplet size of the water phase of the water-in-oil emulsion is no greater than 0.1 μm.

26. Use of a composition as defined in any one of claims 1 to 9 to prevent growth of micro-organisms in an oil and water mixture.

27. Use of a composition as defined in any one of claims 1 to 9 as a fuel oil.

28. A composition as substantially herein before described with reference to the Examples.

29. An emulsion as substantially herein before described with reference to the Examples.

30. A process as substantially herein before described with reference to the Examples.

31. A use as substantially herein before described with reference to the Examples.
